(19) United States
(12) Patent Application Publication
(10) Pub. No.: US 2011/0088695 A1
(43) Pub. Date: Apr. 21, 2011

(54) PROTECTIVE SHEATH FOR USE WITH VENTILATION BAG

(75) Inventor: Samuel DeMaria, JR., New York, NY (US)

(73) Assignee: Mount Sinai School of Medicine, New York, NY (US)

(21) Appl. No.: 12/900,222

(22) Filed: Oct. 7, 2010

Related U.S. Application Data
(60) Provisional application No. 61/251,880, filed on Oct. 15, 2009.

(51) Int. Cl. A61M 16/01

(52) U.S. Cl. 128/205.13

(57) ABSTRACT

In accordance with one embodiment of the present invention, a protective sheath for use with a manual ventilation bag that is associated with an anesthesia apparatus includes a sheath body that is intended to surround the manual ventilation bag. The sheath body has a plurality of removable overlying sheath layers, with each sheath layer being progressively longer in length from an innermost layer to an outermost layer with respect to the ventilation bag that is received within the sheath body.

### CROSS REFERENCE TO RELATED APPLICATIONS

The present application claims the benefit of U.S. patent application Ser. No. 61/251,880, filed Oct. 15, 2009 which is hereby incorporated by reference in its entirety.

### TECHNICAL FIELD

The present invention relates to an anesthesia ventilation system used during surgical operations where anesthesia is delivered to a patient and in particular, the present invention relates to a barrier between a contaminated anesthesia workspace and the practitioner that serves to reduce healthcare associated infection (HCAI) rates.

### BACKGROUND

Anesthesia apparatuses generally are designed to operate in many different operating modes. One operating mode is a manual ventilation mode, in which the anesthesist controls/supports the breathing of a patient by means of a manual ventilation bag. Another operating mode is a mechanical ventilation mode in which the breathing of the patient is controlled/supported by means of a mechanical ventilator. The mechanical ventilation can itself be divided in to a number of sub-modes, such as pressure regulation, volume regulation, etc.

The set-up of an operating mode generally is done via a user interface which itself can include several components located at different parts of the anesthetic device. Parametric values for the different operating modes can also be entered via the user interface.

During manual ventilation, the operator usually is located close to the patient and with one hand can control a facemask on the patient and with the other controls the manual ventilation bag. This usually occurs during the course of administrating the anesthetic.

Before operation, sterile anesthesia equipment, including a sterile breath circuit that includes a breathing tube, adaptor and a manual breathing bag, is delivered to the operating room and connected. Unfortunately, during the normal course of applying the anesthesia, the anesthesiologist often is required to place his or her gloved hand into the patient's mouth for manipulation thereof. This causes a contamination of the gloved hand and when the anesthesiologist then touches the ventilation bag, the bag is contaminated. This results in a contamination of the anesthesia equipment. It is not possible to disconnect and replace the breathing circuit since this would require a disruption in the administration of the anesthesia.

Patient safety has long been a major focus of the field of anesthesiology. While the traditional focus of patient safety for anesthesiologists has included airway device and anesthesia delivery machine-related issues, the role of anesthesiologists in infection control has recently been included. The Centers for Disease Control and the Centers for Medicare and Medicaid Services have both identified the reduction of healthcare associated infections (HCAIs) as a major priority. Currently, 10% of hospitalized patients in the United States acquire an HCAI, contributing to the mortality of 90,000 hospitalized patients and incurring nearly $5 billion in additional healthcare costs annually according to recent studies. Processes such as appropriate and timely antibiotic administration, maintenance of normothermia and more recently, hand hygiene, have been identified as crucial to the reduction of unacceptably high HCAI rates. Given their close contact with perioperative patients and the high likelihood of contamination due to rapid patient care and frequent contact with potential sources of bacterial transmission, anesthesiologists are logical agents of infection control.

Numerous studies have concluded that adherence to hand hygiene guidelines leads to a decrease in microbial transmission and related HCAIs. In one before-and-after study, the introduction of a point of care alcohol-based hand hygiene device led to fewer cases of IV stopcock contamination (from 32% to 8%) and the dramatic reduction in HCAIs (17% to 4%). Anesthesia providers, who have been shown to have poor rates of hand hygiene adherence, are often limited in their options for hand hygiene as most operating rooms may not contain sinks or gel alcohol products. Also, the high workload of anesthesiologists (e.g., during the induction and emergence phases) may lead practitioners to sacrifice hand hygiene for the benefit of more “important” tasks (e.g., airway management). Therefore, early transmission likely occurs after contamination of a provider's hands during the induction of anesthesia. While making hand hygiene measures more accessible is one way around these obstacles, making the anesthesia environment less contaminated is a complementary step.

Hand hygiene is a key measure in reducing HCAIs, yet the importance of the anesthesia workspace as a continued source from which bacterial pathogens may be introduced into patients has not received much attention. In fact, relatively little is known about the anesthesia workspace including the anesthesia machine, cart and intravascular devices. Recently, intraoperative bacterial contamination of the workspace and patient intravenous tubing with pathogens was demonstrated and shown to be associated with an increase in overall patient mortality. Clearly, hand hygiene is crucial but a recently cleaned hand may become contaminated when it touches a colonized surface. For this reason, gloves and hand hygiene are unlikely to reduce the colonization and therefore infection rate, to zero.

Three well-established techniques are important in preventing infectious organism transmission from the provider to the patient: aseptic practice, hand hygiene and barrier techniques. While traditional barrier techniques (e.g., gown and gloves) are well recognized, there is a need for a new set of barrier techniques for the anesthesiologist. More particularly, there is a need for a barrier between a contaminated anesthesia workspace and the practitioner that will lead to a reduction in HCAI rates.

### SUMMARY

In accordance with one embodiment of the present invention, a protective sheath for use with a manual ventilation bag that is associated with an anesthesia apparatus includes a sheath body that is intended to surround the manual ventilation bag. The sheath body has a plurality of removable overlying sheath layers, with each sheath layer being progressively longer in length from an innermost layer to an outermost layer with respect to the ventilation bag that is received within the sheath body.

In another embodiment of the present invention, a ventilation bag assembly for use with an anesthesia apparatus includes a manual ventilation bag having an open first end and a closed second end and a hollow rigid adapter for coupling the ventilation bag to a tube that is part of a breathing circuit. The open first end of the ventilation bag is coupled to an exterior surface of the adapter. The assembly further includes a protective sheath that surrounds the manual ventilation bag and provides an anti-microbial barrier over the ventilation bag. The protective sheath is formed of a plurality of sheath layers that are arranged in an overlying relationship, wherein each sheath layer has a perforated section to permit removal of an outermost exposed sheath layer with respect to the underlying sheath layers.

These and other aspects, features and advantages shall be apparent from the accompanying Drawings and description of certain embodiments of the invention.

### BRIEF DESCRIPTION OF THE DRAWING FIGURES

FIG. 1 is a schematic block diagram of an anesthesia apparatus according to one embodiment of the present invention;

FIG. 2 is a side elevation view of a conventional manual ventilation bag;

FIG. 3 is a side elevation view of a manual ventilation bag according to one embodiment of the present invention;

FIG. 4 is a cross-sectional view taken along the line 4-4 of FIG. 3;

FIG. 5 is a close-up side elevation view of a bottom portion of the ventilation bag of FIG. 3;

FIG. 6 is a close-up side elevation view of a top portion of the bag of FIG. 3; and

FIG. 7 is a side elevation view of one sheath layer showing a perforated section thereof.

### DETAILED DESCRIPTION OF CERTAIN EMBODIMENTS

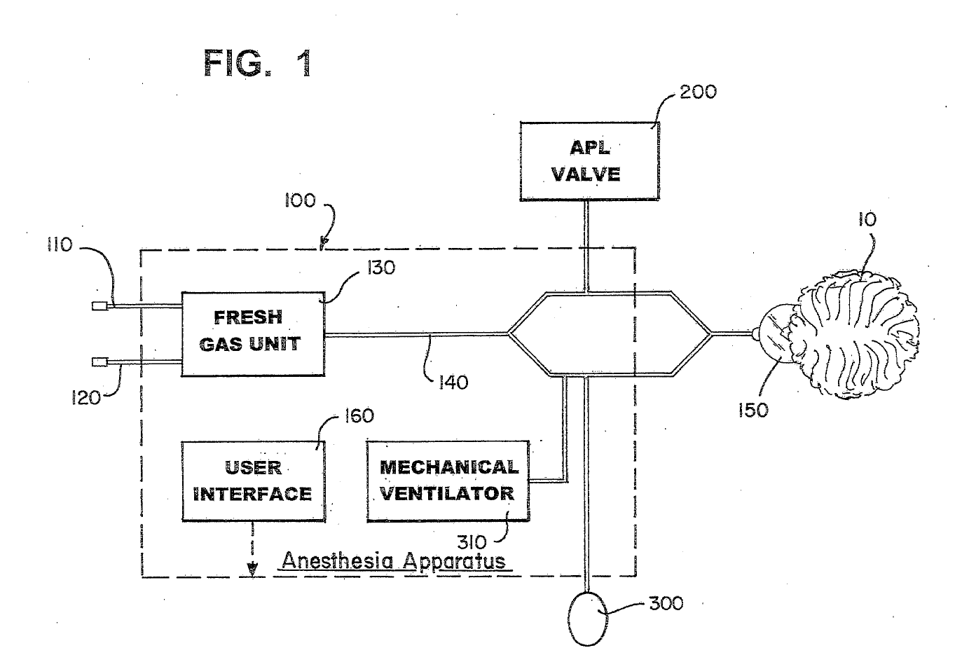

FIG. 1 is a schematic block diagram of an anesthesia apparatus 100 according to one embodiment of the present invention. The anesthetic device 100 is operatively connected to a patient 10 to be anaesthetized. The apparatus 100 includes a number of components for the preparation of fresh gas in the apparatus 100. For example, the apparatus 100 can include a first gas connection 110 for receiving a first gas, such as nitrous oxide, a second gas connection 120 for receiving a second gas, such as oxygen. The apparatus 100 also includes a fresh gas unit 130 for the blending of a chosen mixture of the first and second gases and possibly even for the supply of an anesthetic agent. Other arrangements for the preparation of fresh gas are known and the anesthesia apparatus 100 can equally as well include one or more of these.

The fresh gas is conducted via a tubing system 140 to a facemask 150 that is placed over the mouth and nose of the patient 10. One will appreciate that several different tubing systems are used in the field of anesthesiology, for example, an open system without re-breathing; a half-open system with partial re-breathing; and a closed system with substantial re-breathing.

An anesthesiologist can set an operating mode for the apparatus 100 as well as parametric values by means of a user interface 160. The user interface 160 is designed so that the user can input different values and operating instructions.

An over-pressure valve 200 is also connected to the tubing system 140. The over-pressure valve 200 opens at a settable over-pressure level and is usually known as an APL-valve.

In one operating mode, the apparatus 100 operates in a manual ventilation mode and is therefore provided with a manual ventilation bag 300 and a mechanical ventilator 310. The connection of these to the tubing system 140 can be made in many different known ways and is therefore not discussed herein in any detail.

It will be appreciated that the apparatus 100 can include other working components and the aforementioned description of the apparatus 100 is merely to provide a brief overview of the apparatus 100.

As shown in FIGS. 1-3, the tubing system 140 includes a tube section 142 that terminates in a distal end 144 that is intended for connection to the ventilation bag 300. The tube section 142 is typically a corrugated circuit tubing that permits the ventilator bag 300 to be properly positioned and manipulated. The distal end 144 is coupled to an adapter 400 that is typically in the form of a hard rubber adapter that is complementary to the tubing circuit 142 and the ventilator bag 300.

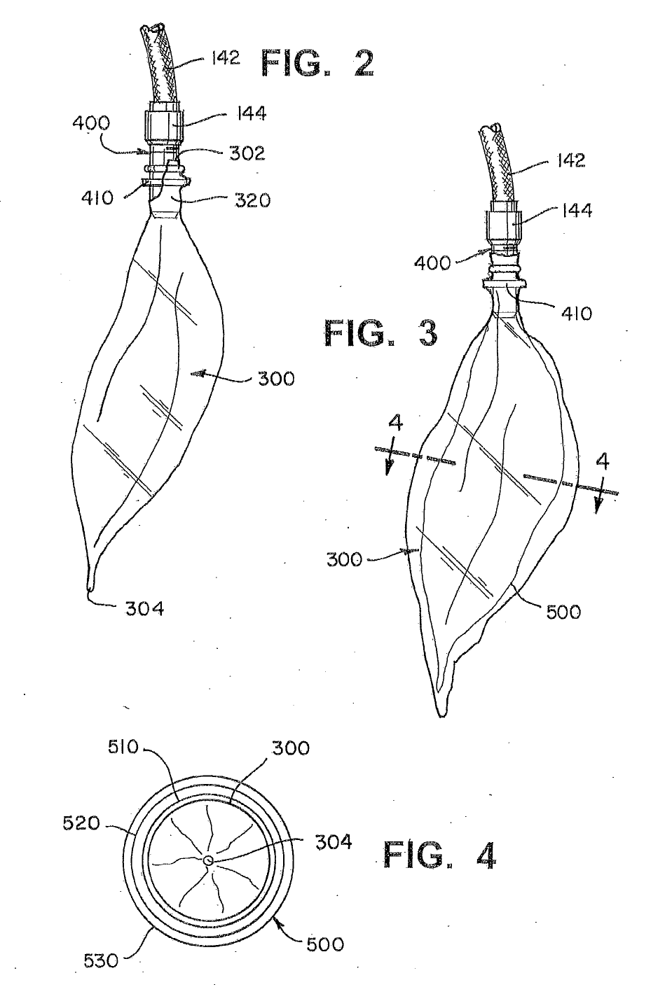

As shown in FIG. 2, a conventional ventilation bag 300 is formed with a neck portion 320 having an opening therethrough which is stretched over the molded adapter 400. The adaptor 400 has a generally cylindrical exterior surface with an outwardly extending flange 410 formed around the periphery at the midpoint thereof. The neck portion 320 of the bag 300 is extended over and beyond the flange 410 where it is secured to the adaptor 400 by the use of a heat shrinkable band. The flange 410 helps to secure the bag 300 to the adaptor 400 and prevents inadvertent slippage of the bag 300 from the adaptor 400. The adaptor 400 is formed so that it can be easily connected to and detached from standard anesthetic systems without the need for other special connectors.

The ventilation bag 300 has a first end 302 at which the neck portion 320 is formed and an opposite second end 304. The first end 302 is thus the end of the ventilation bag 300 that is attached to the adapter 400 and the tube section 142. The opposite second end 304 has a tapered construction in that the second end 304 generally forms a tip or rounded end.

The ventilation bag 300 is typically formed of a rubber material, such as a soft rubber material.

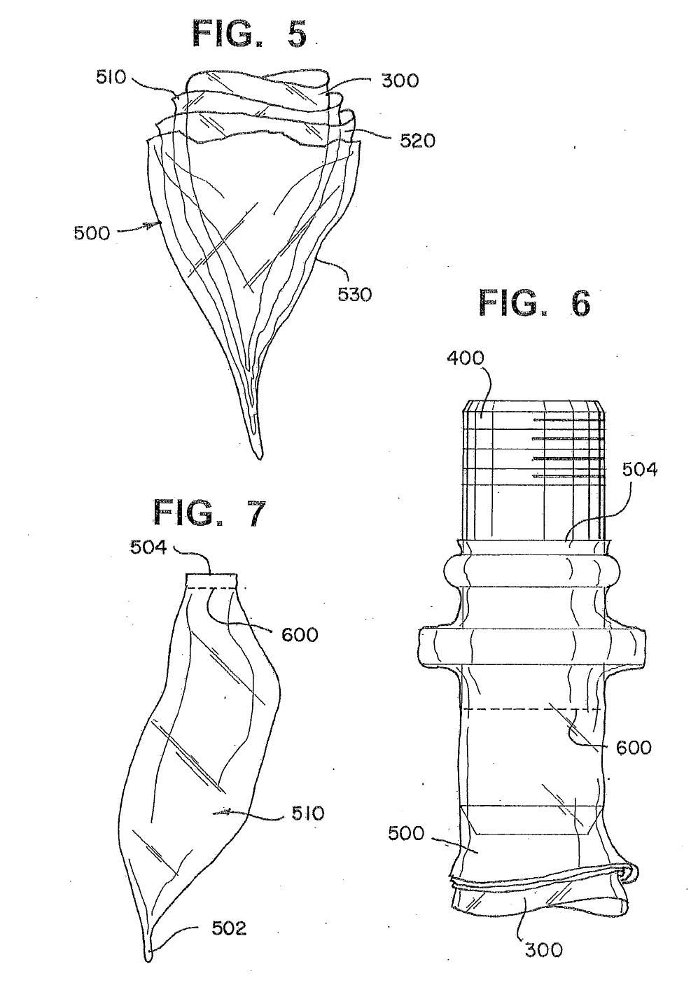

Now referring to FIGS. 3-6, in accordance with the present invention, a barrier 500 is provided over the ventilation bag 300 on the anesthesia circuit for reducing intraoperative bacterial transmission and associated post-operative HCAI rates.

In one embodiment, the barrier 500 is pre-attached to the ventilation bag 300 and distributed in this form. Thus, the bag 300 can be supplied to end users and is fully compliant with existing anesthesia equipment and is simply attached to the breathing circuit in the same manner that a conventional bag 300 is attached.

The barrier 500 is designed to be at least in part removably coupled to the bag 300 so as to provide an anti-microbial sheath for creating a barrier between a contaminated anesthesia workplace and the practitioner. In one embodiment, the barrier 500 is in the form of a multi-layer protective sheath. For example, the barrier 500 can be in the form of a sheath that is formed of three (3) layers as shown best in FIG. 4. The barrier 500 thus has a first sheath layer 510, a second sheath layer 520 and a third sheath layer 530. The first sheath layer 510 is an innermost sheath layer that is closest to the bag 300, the second sheath layer 520 represents an intermediate layer and the third sheath layer 530 represents an outermost sheath layer.

The three sheath layers 510, 520, 530 have similar/identical shapes with the one difference being that the layers are of different sizes. More specifically, each sheath layers 510, 520, 530 is progressively longer from inside to out with respect to the ventilation bag 300. Consequently, the outer sheath 530 has the greatest length, while the inner sheath 510 has the shortest length.

In accordance with the present invention, the size of the barrier 500 and in particular, of each sheath layer 510, 520, 530 depends on the size of the ventilation bag 300 at full inflation. The size of the barrier 500 is such that it extends at least a predetermined distance beyond the distal end 304 of the bag 300 (when fully inflated). In one embodiment, the predetermined distance is at least about 1 inch and therefore, there is about 1 inch of sheath overhand between the innermost sheath layer 510 and the second end 502 to permit the user to grasp the distal end of the sheath layer for removal thereof as described below. The length of the overhang of the other layers 520, 530 is therefore greater than the length of the overhang of the innermost layer 510. FIG. 4 best shows the arrangement of the overhang of the layers 510, 520, 530.

The barrier (protective sheath) 500 is in the form of a non-porous plastic sheath. The protective sheath 500 can have a non-smooth, textured surface which will prevent a gloved-hand from slipping during handling of the sheath 500. The color of the sheath 500 is typically opaque/clear.

The barrier 500 is coupled to a base structure in a manner that causes the protective sheath 500 to surround the bag 300 as illustrated. For example, perforations 600 can be formed in the sheath 500 (e.g., in each layer 510, 520, 530) at or near the proximal end of the barrier 500. More specifically, the free proximal end of the barrier 500 can be coupled to the bag 300 using conventional suitable techniques, including the use of an adhesive. The perforations 600 are thus formed in each sheath layer at a location that surrounds the hard rubber adaptor 400 that is coupled to the ventilation bag 300. The perforations 600 allow each individual sheath layer 510, 520, 530 to tear away easily to reveal the next underlying sheath layer. In this manner, the user simply grabs the overhanging bottom portion of the exposed sheath layer and simply pulls down, thereby causing a rupturing along the perforations 600 and causing the detachment of the sheath layer.

FIG. 7 illustrates a single sheath layer 510 that includes a closed distal end 502 and an open proximal end 504. The perforations (perforated section) 600 is located slightly spaced from the end 504. The end 504 is securely coupled to the bag 300 are in the case of the innermost sheath layer or the proximal end 504 of the other sheath layers can be attached to the underlying sheath layer. In this manner, when the exposed sheath layer is pulled, the perforated section 600 thereof tears, thereby permitting the removal of the sheath layer. It will be understood that a small section of the sheath layer (e.g., the section from the perforations 600 to the end 502) may remain attached to the bag 300 and this is appropriate and does not result in a contamination risk since this end of the sheath layer is unlikely to be manipulated (handled).

It will be appreciated that the bag 300 can be attached to the adaptor 400 using conventional suitable techniques, including the use of an adhesive or the use of a heat seal, etc.

The barrier 500 and in particular, the removable sheath layers 510, 520, 530 thus serve to decrease microbial transmission and related HCAIs by providing a barrier between the ventilator bag 300 which is a potential source of microbial transmission and the practitioner that is in repeated contact with the ventilator bag 300 during the administration of anesthesia to a patient.

As mentioned earlier, as the operation proceeds, it is highly likely that the anesthesiologist will touch a contaminated object (e.g., patient's mouth) and then subsequently need to touch and manipulate the ventilation bag 300. As a result of having the integral protective sheath barrier, the anesthesiologist simply discards the exposed sheath layer that was touched and possibly contaminated. The newly exposed sheath layer is contamination free. As further manipulation of the bag 300 is required and the anesthesiologist contacts the contaminated sites, such as the patient's mouth, the anesthesiologist simply has to discard the exposed sheath layer that is touched after being in contact with a potentially contaminated site. After the operation is complete, the breathing circuit, including the bag 300 and any remaining sheaths are merely discarded.

### Example

Operating rooms in the main operative space at the Mount Sinai Hospital (i.e., the Guggenheim Pavilion, 3^{rd} Floor) were chosen at random by a computer generated list over a period of 6 months and assigned to one of two groups; 1) the non-barrier device group (NB group) and 2) the barrier-device or “bag-in-a-bag” group (BIB group). A separate control group of ten ORs is used wherein the anesthesia environment (i.e., agent dial, manual ventilation bag and APL valve) will be sampled immediately after decontamination. The first case of the day for a chosen OR will be used to avoid potential case-to-case transmission.

The NB group rooms are decontaminated by standard institutional protocols prior to the beginning of the case and are altered in any way by the study group. The attending and/or resident anesthesiologists in the NB group are informed that a study group member will enter the room 30 minutes following the induction of anesthesia and during wound closure to perform bacterial swabs of the environment and of IV stopcock lumens.

The BIB group rooms are decontaminated by standard institutional protocols prior to the beginning of the case. BIB group anesthesia providers are instructed on the use of the barrier device (“BIB”) by a study group member. This member will be available by pager if any device support is needed during the case. A “BIB” is placed over the bag prior to the start of the case by a study group member. Thirty minutes after anesthetic induction, a study group member enters the OR, remove the sheath and perform cultures as in the NB group. During wound closure they return and repeat the cultures.

For both groups, each patient receives a sterile IV tubing and two-stopcock sets are placed preoperatively by anesthesia providers working in a particular OR. If a patient requires two IV's, practitioners are asked to use only one of the sets for injections and that set will ultimately be cultured. If the other set are used for patient or case-related reasons, a study group member will note this.

Laboratory Investigations:
Environment sampling: All cultures are obtained using sterile polyester fiber-tipped applicator swabs moistened with sterile transport medium to roll over the surface twice. The swabs are used to culture on sheep blood agar in a zigzag pattern and swab rotation. Gram positive and negative bacteria are delineated in the microbiology lab using standard procedures.
Stopcock sampling: A sterile nasopharyngeal swab is moistened with sterile transport medium and inserted into the internal port of the open stopcock lumens and rotated 360 degrees ten times. The swab is then used to culture blood agar plates as with the environmental sampling.
Culture conditions: All blood agar plates are incubated at 35 degrees Celsius for 48 hours. Microorganisms are quantified according to cells per surface sampled (CPSS) and identified by standard laboratory methods.

Demographic Data and HCAI Analysis:

Information regarding the patient's age, gender, and ASA status is recorded. Case information including emergent/non-emergent, location status (inpatient, DAS, ambulatory), operative duration and type of procedure are also recorded. Postoperative location is examined (home, inpatient floor or ICU). Practitioner level of experience of the person in the room the longest (e.g., resident vs. attending physician) is also recorded. A computerized medical record is reviewed over 30 postoperative days for evidence of HCAI development and any associated morbidity and mortality in both groups. Reviewers of postoperative data are blinded to culture positivity status and group status.

The present invention provides a number of advantages and is intended to provide a barrier that successfully decreases the level of HCAI and otherwise improves the sanitary conditions in the OR.

While the invention has been described in connection with certain embodiments thereof, the invention is capable of being practiced in other forms and using other materials and structures. Accordingly, the invention is defined by the recitations in the claims appended hereto and equivalents thereof.

What is claimed is:

1. A protective sheath for use with a manual ventilation bag that is associated with an anesthesia apparatus comprising:
a sheath body that is intended to surround the manual ventilation bag, the sheath body having a plurality of removable overlying sheath layers, with each sheath layer being progressively longer in length from an innermost layer to an outermost layer with respect to the ventilation bag that is received within the sheath body.

2. The protective sheath of claim 1, wherein the sheath body has a closed bottom end and an open top end that has a sufficient width to receive the ventilation bag.

3. The protective sheath of claim 1, wherein the sheath body has a length that is greater than a length of the ventilation bag in a fully inflated condition and such that the sheath body extends at least about 1 inch below a closed bottom end of the ventilation bag.

4. The protective sheath of claim 3, wherein each sheath layer extends at least about 1 inch below the closed bottom end of the ventilation bag.

5. The protective sheath of claim 2, wherein the open top ends of the sheath layers are aligned with one another to permit coupling to the ventilation bag associated with a breathing circuit.

6. The protective sheath of claim 1, wherein each sheath layer has a perforated section to permit removal of an outermost sheath layer with respect to the underlying layers.

7. The protective sheath of claim 6, wherein the perforated sections of the plurality of sheath layers are at least substantially aligned with one another and are disposed in an overlying relationship.

8. The protective sheath of claim 1, wherein each sheath layer has a textured outer surface for preventing a gloved-hand from slipping during removal of the sheath layer.

9. The protective sheath of claim 1, wherein the sheath body is formed on a non-porous plastic.

10. The protective sheath of claim 1, wherein the sheath body is formed of three sheath layers that are arranged in an overlying manner.

11. The protective sheath of claim 1, wherein a shape of the sheath layer mirrors a shape of the ventilation bag.

12. A ventilation bag assembly for use with an anesthesia apparatus comprising:
a manual ventilation bag having an open first end and a closed second end, the open first end being adapted for coupling to a hollow rigid adapter that is part of a breathing circuit; and
a protective sheath that surrounds and is securely attached to the manual ventilation bag and provides an anti-microbial barrier over the ventilation bag, the protective sheath formed of a plurality of sheath layers that are arranged in an overlying relationship, wherein each sheath layer has a perforated section proximate an open end thereof to permit removal of an outermost exposed sheath layer with respect to the underlying sheath layers.

13. A disposable breathing circuit for use in an anesthesia system comprising:
a manual ventilation bag having an open first end and a closed second end;
a hollow rigid adapter for coupling the ventilation bag to a tube that is part of a breathing circuit, wherein the open first end of the ventilation bag is coupled to an exterior surface of the adapter; and
a protective sheath that surrounds the manual ventilation bag and provides an anti-microbial barrier over the ventilation bag, the protective sheath formed of a plurality of sheath layers that are arranged in an overlying relationship, wherein each sheath layer has a perforated section to permit removal of an outermost exposed sheath layer with respect to the underlying sheath layers.

14. The breathing circuit of claim 13, wherein each sheath layer is progressively longer in length from an innermost layer to an outermost layer with respect to the ventilation bag that is received within the sheath body.

15. The breathing circuit of claim 13, wherein the sheath body has a length that is greater than a length of the ventilation bag in a fully inflated condition and such that the sheath body extends at least about 1 inch below a closed bottom end of the ventilation bag.

16. The breathing circuit of claim 13, wherein each sheath layer extends at least about 1 inch below the closed bottom end of the ventilation bag.

17. The breathing circuit of claim 13, wherein the open top ends of the sheath layers are aligned with one another to permit coupling to at least one of the ventilation bag and the adaptor associated with the breathing circuit.

18. The breathing circuit of claim 13, wherein the perforated sections of the plurality of sheath layers are at least substantially aligned with one another and are disposed in an overlying relationship and surround the adaptor.

19. The breathing circuit of claim 13, wherein each sheath layer has a textured outer surface for preventing a gloved-hand from slipping during removal of the sheath layer.
